(19) United States
(12) Patent Application Publication
(10) Pub. No.: US 2008/0282364 A1
(43) Pub. Date: Nov. 13, 2008

(54) Phosphodiesterase 9 Inhibition as Treatment for Obesity-Related Conditions

(76) Inventors: Shawn C Black, Ivoryton, CT (US); Earl M Gibbs, Oakdale, CT (US); John D McNeish, Salem, CT (US)

Correspondence Address: PFIZER INC., PATENT DEPARTMENT, MS8260-1611, EASTERN POINT ROAD, GROTON, CT, 06340, US

(21) Appl. No.: 10/577,700

(22) PCT Filed: Oct. 18, 2004
(86) PCT No.: PCT/IS04/03396

§ 371 Date: Apr. 28, 2006

Related U.S. Application Data
(60) Provisional application No. 60/516,213, filed on Oct. 31, 2003.

(51) Int. Cl. A01K 67/027; C12N 5/10; C12N 15/85

(52) U.S. Cl. 800/18; 435/354; 435/366; 800/22

(57) ABSTRACT

The present invention is directed to methods to decrease body weight and/or body fat in an animal, e.g., in the treatment of overweight or obese patients (e.g., humans or companion animals), or as a means to produce leaner meat in food stock animals (e.g., cattle, chickens, pigs), and methods to treat eating disorders (e.g., binge eating disorder and bulimia) in patients in need thereof by administering a PDE9 inhibitor. The invention also features biological tools to further study PDE9 function, i.e., genetically-modified mice and animal cells having a PDE9 gene disruption.

### FIELD OF THE INVENTION

The present invention provides methods to decrease body weight and/or body fat in the treatment, for example, of overweight or obese patients, and methods to treat eating disorders (e.g., binge eating disorder and bulimia), by administering a phosphodiesterase 9 (PDE9) inhibitor. The invention also features genetically-modified mammalian cells, and genetically-modified mice, with a disruption of the PDE9 gene.

### BACKGROUND

Individuals diagnosed as obese or overweight suffer increased risk for developing other health conditions such as coronary heart disease, stroke, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and impart a significant economic and psychosocial effect on society (see, e.g., McGinnis and Foege, JAMA 270: 2207-2212, 1993).

Obesity has become a major public health concern because of its increasing prevalence, and it is now recognized as a chronic disease that requires treatment to reduce its associated health risks. Although weight loss itself is an important treatment outcome, one of the main goals of obesity management is to improve cardiovascular and metabolic values to reduce obesity-related morbidity and mortality. It has been shown that 5-10% loss of body weight can substantially improve metabolic values, such as blood glucose, blood pressure, and lipid concentrations. Hence, it is believed that a 5-10% intentional reduction in body weight may reduce morbidity and mortality.

Cyclic nucleotide phosphodiesterases (PDEs) catalyze the hydrolysis of cyclic nucleotides, such as the second messengers cAMP (cyclic adenosine 3′5′-monophosphate) and cGMP (cyclic guanine 3′5′-monophosphate). Thus, PDEs play a pivotal regulatory role in a wide variety of signal transduction pathways (Beavo, Physiol. Rev. 75: 725-748, 1995). For example, PDEs mediate processes involved in vision (McLaughlin et al., Nat. Genet. 4: 130-134, 1993), olfaction (Yan et al., Proc. Natl. Acad. Sci. USA 92: 9677-81, 1995), platelet aggregation (Dickinson et al. Biochem. J. 323: 371-377, 1997), aldosterone synthesis (MacFarland et al., J. Biol. Chem. 266: 136-142, 1991), insulin secretion (Zhao et al., J. Clin. Invest. 102: 869-873, 1998), T cell activation (Li et al., Science 283: 848-51, 1999), and smooth muscle relaxation (Boolell et al., Int. J. Impot. Res. 8: 47-52, 1996; Ballard et al., J. Urol. 159: 2164-171, 1998).

PDEs form a superfamily of enzymes that are subdivided into 11 major gene families (Beavo, Physiol. Rev. 75: 725-748, 1995; Beavo et al., Mol. Pharmacol. 46: 399-405, 1994; Soderling et al., Proc. Natl. Acad. Sci. USA 95: 8991-8996, 1998; Fisher et al., Biochem. Biophys. Res. Commun. 246: 570-577, 1998; Hayashi et al., Biochem. Biophys. Res. Commun. 250: 751-756, 1998; Soderling et al., J. Biol. Chem. 273: 15553-58, 1998; Fisher et al., J. Biol. Chem. 273: 15559-15564, 1998; Soderling et al., Proc. Natl. Acad. Sci. USA 96: 7071-7076, 1999; and Fawcett et al., Proc. Natl. Acad. Sci. USA 97: 3702-3707, 2000).

Each PDE gene family encodes a phosphodiesterase distinguished functionally by unique enzymatic characteristics and pharmacological profiles. In addition, each family exhibits distinct tissue, cell, and subcellular expression patterns (Beavo et al., Mol. Pharmacol. 46: 399-405, 1994; Soderling et al., Proc. Natl. Acad. Sci. USA 95: 8991-8996, 1998; Fisher et al., Biochem. Biophys. Res. Commun. 246: 570-577, 1998; Hayashi et al., Biochem. Biophys. Res. Commun. 250: 751-756, 1998; Soderling et al., J. Biol. Chem. 273: 15553-15558, 1998; Fisher et al., J. Biol. Chem. 273: 15559-15564, 1998; Soderling et al., Proc. Natl. Acad. Sci. USA 96: 7071-7076, 1999; Fawcett et al., Proc. Natl. Acad. Sci. USA 97: 3702-3707, 2000; Boolell et al., Int. J. Impot. Res. 8: 47-52, 1996; Ballard et al., J. Urol. 159: 2164-71, 1998; Houslay, Semin. Cell Dev. Biol. 9: 161-67, 1998; and Torphy et al., Pulm. Pharmacol. Ther. 12: 131-135, 1999). Therefore, by administering a compound that selectively regulates the activity of one family or subfamily of PDE enzymes, it is possible to regulate cAMP and/or cGMP signal transduction pathways in a cell- or tissue-specific manner.

Fisher et al. (J. Biol. Chem. 273: 15559-15564, 1998) identified the PDE9 enzyme as a novel member of the PDE enzyme family that selectively hydrolyses cGMP over cAMP. PDE9 is present in a variety of human tissues, including testes, brain, small intestine, skeletal muscle, heart, lung, thymus, and spleen. PDE9 inhibitors have bee reported as useful to treat cardiovascular disorders (WO 03/037899), and insulin resistance syndrome, hypertension, and/or type 2 diabetes (WO 03/037432).

### SUMMARY OF THE INVENTION

In a first aspect, the invention features a method of treating an animal to reduce body fat comprising administering to an animal in need thereof a therapeutically effective amount of a PDE9 inhibitor. Preferably, the animal is a human or companion animal (e.g., dog, cat, horse) and is overweight, more preferably, the animal is obese. In another preferred embodiment, the animal is a food stock animal (e.g., chicken, cattle, pig) and such treatment is rendered to produce leaner meat. In another preferred embodiment, the PDE9 inhibitor is a PDE9 selective inhibitor or the PDE9 inhibitor is administered orally.

In a second aspect, the invention features a method of treating an animal for an eating disorder comprising administering to an animal in need thereof a therapeutically effective amount of a PDE9 inhibitor. Preferably, the eating disorder is binge eating disorder or bulimia, the PDE9 inhibitor is a PDE9 selective inhibitor, or the PDE9 inhibitor is administered orally.

In a third aspect, the invention features a method of treating an animal for metabolic syndrome comprising administering to an animal in need thereof a therapeutically effective amount of a PDE9 inhibitor. Preferably, the PDE9 inhibitor is a PDE9 selective inhibitor, or the PDE9 inhibitor is administered orally.

The invention also features a genetically-modified mouse, wherein the mouse is homozygous for disruption of the PDE9 gene and wherein the mouse, following a six week high fat diet, exhibits reduced body weight or reduced fat mass in an adipose depot, as compared to a wild type mouse following a six week high fat diet. In a preferred embodiment, the mouse expresses an exogenous reporter gene under the control of the regulatory sequences of the PDE9 gene or the mouse exhibits nondetectable PDE9 activity. In a related aspect, the invention provides a cultured genetically-modified murine cell derived from the above-described mouse. In another related aspect, the invention provides a method for producing the above-described mouse comprising: (a) introducing a DNA sequence into a mouse ES cell, wherein the DNA sequence comprises a PDE9 gene targeting construct, which, upon recombination with the PDE9 gene, disrupts the PDE9 gene; (b) selecting a mouse ES cell whose genome comprises a disruption of the PDE9 gene; (c) introducing an ES cell selected in step (b) into a mouse blastocyst or morulae; (d) transplanting the blastocyst or morulae of step (c) into a pseudopregnant mouse; (e) developing the transferred blastocyst or morulae to term to produce a chimeric mouse; and (f) mating sexually mature chimeric mice and mice heterozygous for the PDE9 disruption to obtain a mouse homozygous for the PDE9 gene disruption; wherein the mouse, following a six week high fat diet, exhibits reduced body weight or reduced fat mass in an adipose depot, as compared to a wild type mouse following a six week high fat diet.

The invention also features a genetically-modified cultured mammalian cell,

wherein the cell is homozygous for disruption of the PDE9 gene and wherein the cell, or a progeny cell derived from the cell, exhibits nondetectable PDE9 polypeptide activity wherein the cell or progeny cell would exhibit PDE9 polypeptide activity absent the homozygous disruption. In a preferred embodiment, the cell is an embryonic stem (ES) cell, more preferably, the cell is a murine ES cell or a human ES cell.

In another aspect, the invention provides an isolated nucleic acid molecule comprising a PDE11 gene targeting construct, wherein, upon recombination with the PDE9 gene, the construct disrupts the PDE9 gene.

Those skilled in the art will fully understand the terms used herein in the description and the appendant claims to describe the present invention. Nonetheless, unless otherwise provided herein, the following terms are as described immediately below.

By “PDE9 inhibitor” is meant an agent that reduces or attenuates the biological activity of the PDE9 polypeptide. Such agents may include proteins, such as anti-PDE9 antibodies, nucleic acids, e.g., PDE9 antisense or RNA interference (RNAi) nucleic acids, amino acids, peptides, carbohydrates, small molecules (organic or inorganic), or any other compound or composition which decreases the activity of a PDE9 polypeptide either by effectively reducing the amount of PDE9 present in a cell, or by decreasing the enzymatic activity of the PDE9 polypeptide. Compounds that are PDE9 inhibitors include all solvates, hydrates, pharmaceutically acceptable salts, tautomers, stereoisomers, and prodrugs of the compounds. Preferably, a small molecule PDE9 inhibitor used in the present invention has an IC₅₀ of less that 10 μM, more preferably, less than 1 μM, and, even more preferably, less than 0.1 μM. Any PDE9 inhibitor used in the present invention is preferably also selective against some or all other PDEs, preferably, against PDE1A, PDE1B, PDE1C, PDE2, PDE3A, PDE3B, PDE4A, PDE4B, PDE4C, PDE4D, PDE5, PDE6, PDE7A, PDE7B, PDE8A, PDE8B, PDE10, and/or PDE11.

By a “selective” PDE9 inhibitor is meant an agent that inhibits PDE9 activity with an IC₅₀ at least 10-fold less, preferably, at least 100-fold less, than the IC₅₀ for inhibition of one or more other PDEs. Preferably, such agents are combined with a pharmaceutically acceptable delivery vehicle or carrier. An antisense oligonucleotide directed to the PDE9 gene or mRNA to inhibit its expression is made according to standard techniques (see, e.g., Agrawal et al. Methods in Molecular Biology: Protocols for Oligonucleotides and Analogs, Vol. 20, 1993). Similarly, an RNA molecule that functions to reduce the production of PDE9 enzyme in a cell can be produced according to standard techniques known to those skilled in the art (see, e.g., Hannon, Nature 418: 244-251, 2002; Shi, Trends in Genetics 19: 9-12, 2003; Shuey et al., Drug Discovery Today 7: 1040-1046, 2002). Examples of PDE9 inhibitors are provided herein and in WO 03/037899, in WO 03/037432, and in U.S. Provisional Patent Appl. No. 60/466,639, filed Apr. 30, 2003, incorporated herein by reference.

“Decreased PDE9 activity” means a manipulated decrease in the polypeptide activity of the PDE9 enzyme as a result of genetic disruption or manipulation of the PDE9 gene function that causes a reduction in the level of functional PDE9 polypeptide in a cell, or as the result of administration of a pharmacological agent that inhibits PDE9 activity.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “prodrug” refers to a compound that is a drug precursor which, following administration, releases the drug in vivo via a chemical or physiological process (e.g., upon being brought to physiological pH or through enzyme activity). A discussion of the synthesis and use of prodrugs is provided by Higuchi and Stella, Prodrugs as Novel Delivery Systems, vol. 14 of the ACS Symposium Series, and Bioreversible Carriers in Drug Design, ed. Edward B. Roche, American Pharmaceutical Association and Pergamon Press, 1987.

The terms “salts” and “pharmaceutically acceptable salts” refer to organic and inorganic salts of a compound, a stereoisomer of the compound, or a prodrug of the compound.

“Overweight” and the more severe “obese” conditions, in an adult person 18 years or older, constitute having greater than ideal body weight (more specifically, greater than ideal body fat) and are generally defined by body mass index (BMI), which is correlated with total body fat and the relative risk of suffering from premature death or disability due to disease as a consequence of the overweight or obese condition. The health risks increase with the increase in excessive body fat. BMI is calculated by weight in kilograms divided by height in meters squared (kg/m²) or, alternatively, by weight in pounds, multiplied by 703, divided by height in inches squared (lbs×703/in²). “Overweight” typically constitutes a BMI of between 25.0 and 29.9. “Obesity” is typically defined as a BMI of 30 or greater (see, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083, 1998). In heavily muscled individuals, the correlation between BMI, body fat, and disease risk is weaker than in other individuals. Therefore, assessment of whether such heavily muscled individuals are in fact overweight or obese may be more accurately performed by another measure such as direct measure of total body fat or waist-to-hip ratio assessment.

By a “high fat diet”, as administered to a genetically-modified or wild type mouse, is meant a diet composed of at least 45% kcal fat, and, preferably, at least 58% fat. Exemplary diets include the Surwit diet (Surwit et al., Metabolism 47: 1354-1359; Surwit et al., Metabolism 47: 1089-1096, 1998; Surwit et al., J. Biol. Chem. 271: 9437-9440, 1996; and Surwit et al., Metabolism 44: 645-651, 1995), D12451 Rodent Diet (45% kcal fat, Research Diets, Inc., New Brunswick, N.J.), and D12331 Rodent Diet (58% kcal fat, Research Diets, Inc.)

“Metabolic syndrome”, as defined herein, and as according to the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. no. 01-3670), occurs when a person has three or more of the following criteria:

1. Abdominal obesity: waist circumference >102 cm in men and >88 cm in women;
2. Hypertriglyceridemia: ≧150 mg/dl (1.695 mmol/l);
3. Low HDL cholesterol: <40 mg/dl (1.036 mmol/l) in men and <50 mg/dl (1.295 mmol/l) in women;
4. High blood pressure: ≧130/85 mmHg;
5. High fasting glucose: ≧1110 mg/dl (≧6.1 mmol/l); or, as according to World Health Organization criteria (Alberti and Zimmet, Diabet. Med. 15: 539-53, 1998), when a person has diabetes, impaired glucose tolerance, impaired fasting glucose, or insulin resistance plus two or more of the following abnormalities:
1. High blood pressure: ≧160/90 mmHg;
2. Hyperlipidemia: triglyceride concentration ≧150 mg/dl (1.695 mmol/l) and/or HDL cholesterol <35 mg/dl (0.9 mmol/l) in men and <39 mg/dl (1.0 mmol/l) in women;
3. Central obesity: waist-to-hip ratio of >0.90 for men and >0.85 in women and/or BMI >30 kg/m²;
4. Microalbuminuria: urinary albumin excretion rate ≧20 μg/min or an albumin-to-creatinine ratio ≧20 mg/kg.

By “therapeutically effective” is meant resulting in a decrease in body fat.

A “disrupted PDE9 gene” refers to a PDE9 gene that is genetically-modified such that the cellular activity of the PDE9 polypeptide encoded by the disrupted gene is decreased or, preferably, eliminated in cells that normally express a wild type version of the PDE9 gene. When the genetic modification effectively eliminates all wild type copies of the PDE9 gene in a cell (e.g., the genetically-modified, non-human mammal or animal cell is homozygous for the PDE9 gene disruption or the only wild type copy of the PDE9 gene originally present is now disrupted), the genetic modification results in a reduction in PDE9 polypeptide activity as compared to a control cell that expresses the wild type PDE9 gene. This reduction in PDE9 polypeptide activity results from either reduced PDE9 gene expression (i.e., PDE9 mRNA levels are effectively reduced resulting in reduced levels of PDE9 polypeptide) and/or because the disrupted PDE9 gene encodes a mutated polypeptide with altered, e.g., reduced, function as compared to a wild type PDE9 polypeptide. Preferably, the activity of PDE9 polypeptide in the genetically-modified, non-human mammal or animal cell is reduced to 50% or less of wild type levels, more preferably, to 25% or less, and, even more preferably, to 10% or less of wild type levels. Most preferably, the homozygous PDE9 gene disruption results in non-detectable PDE9 activity in cells of a type that demonstrate wild type PDE9 activity.

A “genetically-modified, non-human mammal” containing a disrupted PDE9 gene refers to a non-human mammal created by genetic engineering to contain a disrupted PDE9 gene, as well as a progeny of such non-human mammal that inherits the disrupted PDE9 gene. A genetically-modified non-human mammal may be produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. Alternatively, various species of genetically-modified embryos can be obtained by nuclear transfer. In the case of nuclear transfer, the donor cell is a somatic cell or a pluripotent stem cell, and it is engineered to contain the desired genetic modification that disrupts the PDE9 gene. The nucleus of this cell is then transferred into a fertilized or parthenogenetic oocyte that is enucleated; the resultant embryo is reconstituted and developed into a blastocyst. A genetically-modified blastocyst produced by either of the above methods is then implanted into a foster mother according to standard methods well known to those skilled in the art. A “genetically-modified, non-human mammal” includes all progeny of the non-human mammals created by the methods described above, provided that the progeny inherit at least one copy of the genetic modification that disrupts the PDE9 gene. It is preferred that all somatic cells and germlne cells of the genetically-modified non-human mammal contain the modification. Preferred non-human mammals that are genetically-modified to contain a disrupted PDE9 gene include rodents, such as mice and rats, cats, dogs, rabbits, guinea pigs, hamsters, sheep, pigs, and ferrets.

A “genetically-modified animal cell” containing a disrupted PDE9 gene refers to an animal cell (preferably a mammalian cell), including a human cell, created by genetic engineering to contain a disrupted PDE9 gene, as well as daughter cells and cells differentiated from a genetically-modified parent ES or stem cell, that inherit the disrupted PDE9 gene. These cells may be genetically-modified in culture according to any standard method known in the art. As an alternative to genetically modifying the cells in culture, non-human mammalian cells may also be isolated from a genetically-modified, non-human mammal that contains a PDE9 gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas, kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, embryonic germline cells, and other stem cells, such as progenitor cells and tissue-derived stem cells). The preferred genetically-modified cells are ES cells, more preferably, mouse or rat ES cells, and, most preferably, human ES cells, as well as cells differentiated from the genetically-modified ES cells.

A non-human mammal or a animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification to disrupt a gene are those which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the PDE9 gene, e.g., in an enhancer, promoter, regulator region, noncoding region, coding region, intron, or exon. The most preferred method of genetic engineering for gene disruption is homologous recombination, in which the foreign nucleic acid sequence is inserted in a targeted manner either alone or in combination with a deletion of a portion of the endogenous gene sequence.

“Homozygosity”, when referring to PDE9 gene disruption in a non-human mammal or an animal cell, means a non-human mammal or animal cell having disruption of all alleles of the PDE9 gene. However, the PDE9 gene sequences of each of these disrupted alleles need not be identical. For example, a non-human mammal may be homozygous for PDE9 disruption wherein one allele of PDE9 is disrupted as a result of deletion of one region of the gene sequence and the other allele is disrupted as a result of deletion of another region of the gene sequence.

“ES cell” or an “ES-like cell” means a pluripotent stem cell derived from an embryo, from a primordial germ cell, or from a teratocarcinoma, that is capable of indefinite self-renewal as well as differentiation into cell types that are representative of all three embryonic germ layers.

“Microarray” means an arrangement of distinct polynucleotides or polypeptides on a substrate, as more fully described herein.

“Wild type”, when referring to a non-human mammal or an animal cell, means a non-human mammal or an animal cell, as the case may be, that does not comprise a disrupted PDE9 gene. For example, in a comparison of a particular characteristic of a non-human mammal of this invention to that characteristic in a wild type mammal, the term wild type refers to non-human mammal that does not comprise a disrupted PDE9 gene (i.e., a mammal whose PDE9 gene is wild type). Preferably, a wild type non-human mammal is substantially similar, and, more preferably, substantially identical, to a non-human mammal of the invention, except for the non-disruption or disruption of the PDE9 gene, respectively. Likewise, for example, in a comparison of a particular characteristic of an animal cell of this invention to that characteristic in a wild type animal cell, the term wild type refers to an animal cell that does not comprise a disrupted PDE9 gene (i.e., a cell whose PDE9 gene is wild type). Preferably, a wild type animal cell is substantially similar, and, more preferably, substantially identical, to an animal cell of the invention, except for the non-disruption or disruption of the PDE9 gene, respectively.

Other features and advantages of the invention will be even further apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y., 1986; Hames et al., Nucleic Acid Hybridization, IL Press, 1985; Molecular Cloning, Sambrook et al., Current Protocols in Molecular Biology, Eds. Ausubel et al., John Wiley and Sons, 2001; Current Protocols in Human Genetics, Eds. Dracopoli et al., John Wiley and Sons, 1994; Current Protocols in Protein Science, Eds. John E. Coligan et al., John Wiley and Sons, 2002; and Current Protocols in Immunology, Eds. John E. Coligan et al., John Wiley and Sons, 1994). All publications, including published patent applications and issued patents, mentioned herein are incorporated by reference in their entireties.

### BRIEF DESCRIPTION OF THE FIGURES

FIG. 1 is a schematic of the targeting construct used to disrupt the PDE9 gene. The 5′ and 3′ homology arms complementary to PDE9 genomic sequence, 0.9 kb and 4.3 kb in length, respectively, flanked a LacZ-Neo cassette. A portion of the genomic sequence of each homology arm is shown as SEQ ID NO: 1 and SEQ ID NO: 2.

FIG. 2 shows the cDNA sequence for a murine PDE9 (SEQ ID NO: 3). Upon homologous recombination with the targeting construct, the underlined sequence, base pairs 142-175, was deleted and replaced with LacZ-Neo.

FIG. 3 is a line graph detailing the body weight change in wild type (WT) and genetically-modified mice homozygous for disruption of the PDE9 gene (PDE9 knockout (KO) mice) during the course of a six week high fat diet.

FIG. 4A (male) and FIG. 4B (female) are bar graphs showing the mass of several adipose depots in WT and PDE9 KO mice after a six week high fat diet. SC—subcutaneous; TBW—total body weight.

FIG. 5 is a bar graph comparing the body weight of female WT and PDE9 KO mice following a six week control chow diet.

FIG. 6A (baseline) and FIG. 6B (post-six week chow diet) are bar graphs showing the mass of adipose depots in female WT and PDE9 KO mice. (Ing—inguinal subcutaneous; Gon—gonadal; RP—retroperitoneal; Mes—mesenteric)

FIG. 7 is a line graph detailing the time course for body weight gain in female ob/ob mice in Control, Compound A-treated (100 mg/kg/day), and Darglitazone-treated groups.

FIG. 8A is a bar graph showing the Compound A dose effect on body weight at Days 2 and 4 in female ob/ob mice. FIG. 8B is a bar graph showing the Compound A dose effect on food consumption at Days 2 and 4 in female ob/ob mice.

FIG. 9 is a bar graph comparing the time course for food consumption between Control, Compound A-treated (100 mg/kg/day), and Darglitazone-treated female ob/ob mice.

FIG. 10 is a line graph comparing plasma glucose in Control, Compound A-treated (100 mg/kg/day), and Darglitazone-treated female ob/ob mice.

FIG. 11 is a line graph showing plasma triglycerides in Control and Compound A-treated (50 and 100 mg/kg/day) female ob/ob mice at Days 1, 2, and 4.

FIG. 12 is a bar graph comparing plasma fructosamine in Control, Compound A (100 mg/kg/day), and Darglitazone-treated female ob/ob mice at Day 16.

### DETAILED DESCRIPTION OF THE INVENTION

The present invention is directed to methods to decrease body weight and/or body fat in an animal, e.g., in the treatment of overweight or obese patients (e.g., humans or companion animals), or as a means to produce leaner meat in food stock animals (e.g., cattle, chickens, pigs), and methods to treat eating disorders (e.g., binge eating disorder and bulimia) in patients in need thereof by administering a PDE9 inhibitor. The invention also features biological tools to further study PDE9 function, i.e., genetically-modified mice and animal cells having a PDE9 gene disruption. As disclosed in the Examples herein, administration of a PDE9 inhibitor reduces weight gain in the ob/ob mouse model of obesity, and PDE9 knockout mice are relatively resistant to developing increased body weight and increased adiposity subsequent to exposure to a high fat diet. Both Examples demonstrate that causing a decrease in PDE9 activity is an effective method to reduce body weight and/or body fat, and can be used, e.g., to treat animal patients that are overweight, obese, or suffer from an eating disorder, and can be used in animal food stock species to produce leaner meat.

### Exemplary PDE9 Inhibitors

Any PDE9 inhibitor may be used in this invention. PDE9 inhibitors are known to those skilled in the art and may be determined by standard assays known to those in the art, such as in WO 03/037899 and WO 03/037432. The PDE9 inhibitors used in the methods of the invention include those disclosed in WO 03/037899 and WO 03/037432, as well as in U.S. Provisional Appl. No. 60/466,639, filed Apr. 30, 2003, incorporated hereinbefore by reference. Compounds disclosed as PDE9 inhibitors in the above-discussed U.S. Provisional Patent Appl. include:
3-isopropyl-5-[2-(2-morpholin-4-yl-ethoxy)-benzyl]-1,6-dihydro-pyrazolo[4,3-d]pyrimidin-7-one (hereinafter referred to as “Compound A”);
1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid;
3-isopropyl-5-[2-(2-oxo-2-piperazin-1-yl-ethoxy)-benzyl]-1,6-dihydro-pyrazolo[4,3-d]pyrimidin-7-one trifluoro acetate;
3-isopropyl-5-[2-(2-morpholin-4-yl-2-oxo-ethoxy)-benzyl]-1,6-dihydro-pyrazolo[4,3-d]pyrimidin-7-one;
3-isopropyl-5-[2-(2-oxo-2-pyrrolidin-1-yl-ethoxy)-benzyl]-1,6-dihydro-pyrazolo[4,3-d]pyrimidin-7-one;
N,N-diethyl-2-[2-(3-isopropyl-7-oxo-6,7-dihydro-1H-pyrazolo[4,3-d]pyrimidin-5-ylmethyl)-phenoxy]-acetamide;
1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid methyl ester;
4-{[2-(3-isopropyl-7-oxo-6,7-dihydro-1H-pyrazolo[4,3-d]pyrimidin-5-ylmethyl)-phenoxy]-acetyl}-piperazine-1-carboxylic acid tert-butyl ester;
N-(2-dimethylamino-ethyl)-2-[2-(3-isopropyl-7-oxo-6,7-dihydro-1H-pyrazolo[4,3-d]pyrimidin-5-ylmethyl)-phenoxy]-acetamide;
[2-(3-isopropyl-7-oxo-6,7-dihydro-1H-pyrazolo[4,3-d]pyrimidin-5-ylmethyl)-phenoxy]-acetic;
3-isopropyl-5-[2-(5-chloro-2-morpholin-4-yl-ethoxy)-benzyl]-1,6-dihydro-pyrazolo[4,3-d]pyrimidin-7-one;
3-isopropyl-5-[2-(2-pyrrolidin-1-yl-ethoxy)-benzyl]-1,6-dihydro-pyrazolo[4,3-d]pyrimidin-7-one;
3-isopropyl-5-[2-(2-morpholin-4-yl-ethoxy)-cyclohexylmethyl]-1,6-dihydro-pyrazolo[4,3-d]pyrimidin-7-one;
5-[5-fluoro-2-(2-morpholin-4-yl-ethoxy)-benzyl]-3-isopropyl-1,6-dihydro-pyrazolo[4,3-d]pyrimidin-7-one;
3-cyclopentyl-5-[5-fluoro-2-(2-morpholin-4-yl-ethoxy)-benzyl]-1,6-dihydro-pyrazolo[4,3-d]pyrimidin-7-one;
9-(1,2-dimethyl-propyl)-2-[2-(2-morpholin-4-yl-ethoxy)-benzyl]-1,9-dihydro-purin-6-one;
2-[2-(2-morpholin-4-yl-ethoxy)-benzyl]-9-(tetrahydro-furan-3-yl)-1,9-dihydro-purin-6-one;
5-[2-(2-diethylamino-ethoxy)-benzyl]-3-isopropyl-1,6-dihydro-pyrazolo[4,3-d]pyrimidin-7-one;
3-cyclopentyl-5-[2-(2-morpholin-4-yl-ethoxy)-benzyl]-1,6-dihydro-pyrazolo[4,3-d]pyrimidin-7-one;
9-(1(R),2-dimethyl-propyl)-[2-(2-morpholin-4-yl-ethoxy)-benzyl]-1,9-dihydro-purin-6-one;
9-(2-methyl-butyl)-2-[2-(2-morpholin-4-yl-ethoxy)-benzyl]-1,9-dihydro-purin-6-one;
9-cyclopentyl-2-[2-(2-morpholin-4-yl-ethoxy)-benzyl]-1,9-dihydro-purin-6-one;
5-[2-(2-morpholin-4-yl-ethoxy)-benzyl]-3-pyridin-3-yl-1,6-dihydro-pyrazolo[4,3-d]pyrimidin-7-one;
9-(1,2-dimethyl-propyl)-2-[2-(2-morpholin-4-yl-ethoxy)-benzyl]-1,9-dihydro-purin-6-one;
9-isopropyl-2-[2-(2-morpholin-4-yl-ethoxy)-benzyl]-1,9-dihydro-purin-6-one;
2-[2-(2-morpholin-4-yl-ethoxy)-benzyl]-9-(tetrahydro-furan-2-ylmethyl)-1,9-dihydro-purin-6-one;
9-(1-isopropyl-2-methyl-propyl)-2-[2-(2-morpholin-4-yl-ethoxy)-benzyl]-1,9-dihydro-purin-6-one;
9-(1-ethyl-propyl)-2-[2-(2-morpholin-4-yl-ethoxy)-benzyl]-1,9-dihydro-purin-6-one; and
N-[2-(3-isopropyl-7-oxo-6,7-dihydro-1H-pyrazolo[4,3-d]pyrimidin-5-ylmethyl)-cyclohexyl]-2-pyrrolidin-1-yl-acetamide.

It will be understood by those skilled in the art that all sterioisomers, tautomers, solvates, hydrates prodrugs, and pharmaceutically acceptable salts of the compounds listed above are also included.

### Therapeutic Methods

An agent identified as a PDE9 inhibitor is administered in a dose sufficient to reduce body weight or body fat, e.g., by reducing the mass of one or more adipose depots. Such therapeutically effective amounts will be determined using routine optimization techniques that are dependent on, for example, the condition of the patient or animal, the route of administration, the formulation, the judgment of the practitioner, and other factors evident to those skilled in the art in light of this disclosure.

The PDE9 inhibitors suitable for use in accordance with the present invention can be administered alone but, in human therapy, will generally be administered in admixture with a suitable pharmaceutical excipient diluent or carrier selected with regard to the intended route of administration and standard pharmaceutical practice.

For example, the PDE9 suitable for use in accordance with the present invention or salts or solvates thereof can be administered orally, buccally or sublingually in the form of tablets, capsules (including soft gel capsules), multi-particulate, gels, films, ovules, elixirs, solutions or suspensions, which may contain flavoring or coloring agents, for immediate-, delayed-, modified-, sustained-, dual-, controlled-release or pulsatile delivery applications. Such compounds may also be administered via fast dispersing or fast dissolving dosages forms or in the form of a high energy dispersion or as coated particles. Suitable pharmaceutical formulations may be in coated or un-coated form as desired.

Such solid pharmaceutical compositions, for example, tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), hydroxypropyl methylcellulose acetate succinate (HPMCAS), sucrose, gelatin and acacia. Additionally, lubricating agents such as magnesium stearate, stearic acid, glyceryl behenate and talc may be included.

Solid compositions of a similar type may also be employed as fillers in gelatin capsules or HPMC capsules. Preferred excipients in this regard include lactose, starch, a cellulose, milk sugar or high molecular weight polyethylene glycols. For aqueous suspensions and/or elixirs, the PDE9 inhibitor compounds may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, HPMC, HPMCAS, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form, i.e., within the matrix, and/or on the dosage form, i.e., upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e., in cases where the drug substance is insoluble, a fast dispersing dosage form can be prepared, and, in cases where the drug substance is soluble, a fast dissolving dosage form can be prepared.

The PDE9 inhibitors suitable for use in accordance with the present invention can also be administered parenterally, for example, intracavernosally, intravenously, intra-arterially, intraperitoneally, intrathecally, intraventricularly, intraurethrally, intrasternally, intracranially, intramuscularly or subcutaneously, or they may be administered by infusion or needle-free techniques. For such parenteral administration they are best used in the form of a sterile aqueous solution which may contain other substances, for example, enough salts or glucose to make the solution isotonic with blood. The aqueous solutions should be suitably buffered (preferably to a pH of from about 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the PDE9 inhibitors for use in the present invention will usually be from 1 to 500 mg (in single or divided doses). A preferred dosage range is about 1 mg to about 100 mg. The dosage may by via single dose, divided daily dose, or multiple daily dose. Alternatively, continuous dosing, such as for example, via a controlled release dosage form wherein such continuous dosage form can be administered on a daily basis or wherein such continuous dosing can be affected via a slow-release formulation which doses for more than one day at a time.

Thus, for example, tablets or capsules of the PDE9 inhibitors suitable for use in accordance with the present invention may contain from 1 mg to 250 mg of active compound for administration singly or two or more at a time, as appropriate. Preferred tablets or capsules will contain about 1 mg to about 50 mg of active compound for administration singly or two or more at a time, as appropriate. The physician in any event will determine the actual dosage which will be most suitable for any individual patient and it will vary with the age, weight and response of the particular patient. There can, of course, be individual instances where higher or lower dosage ranges are merited and such are within the scope of this invention.

The PDE9 inhibitors suitable for use in accordance with the present invention can also be administered intranasally or by inhalation and are conveniently delivered in the form of a dry powder inhaler or an aerosol spray presentation from a pressurized container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a PDE9 inhibitor for delivery to the animal to be treated. The overall daily dose with an aerosol will be in the range of from 1 to 50 mg which may be administered in a single dose or, more usually, in divided doses throughout the day.

The PDE9 inhibitors suitable for use in accordance with the present invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the PDE9 inhibitors suitable for use in accordance with the present invention can be administered in the form of a suppository or pessary, or they may be applied topically in the form of a gel, hydrogel, lotion, solution, cream, ointment or dusting powder. The PDE9 inhibitors suitable for use in accordance with the present invention may also be dermally or transdermally administered, for example, by the use of a skin patch. They may also be administered by the pulmonary or rectal routes.

The PDE9 inhibitors may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the PDE9 inhibitors suitable for use in accordance with the present invention can be formulated as a suitable ointment containing the active ingredient or agent suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The PDE9 inhibitors suitable for use in accordance with the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are some of the most commonly used and suitable examples are described in WO 91/11172, WO 94/02518 and WO 98/55148.

Generally, in humans, oral administration is the preferred route, being the most convenient. In circumstances where the recipient suffers from a swallowing disorder or from impairment of drug absorption after oral administration, the drug may be administered parenterally, sublingually, or buccally.

For veterinary use, a PDE9 inhibitor is administered as a suitably acceptable formulation in accordance with normal veterinary practice and the veterinary surgeon will determine the dosing regimen and route of administration which will be most appropriate for a particular animal. Such animals include companion animals who are overweight, obese, or at risk of being overweight or obese. Other animals that may be treated according to the present invention are foodstock animals in order to obtain leaner meat than would be obtained absent treatment according to the present invention.

Therapeutic efficacy of such PDE9 inhibitors can be determined in light of this disclosure by standard therapeutic procedures in cell cultures or experimental animals, e.g., for determining the ED₅₀ (the dose therapeutically effective in 50% of the population).

The data obtained from the cell culture assays and animal studies can be used in formulating a range of dosage for use in humans. The dosage may vary, for example, depending upon the formulation and the route of administration. For any PDE9 inhibitor used in the method of the invention, the therapeutically effective dose can be estimated initially from cell culture assays. A dose may be formulated in animal models to achieve a circulating plasma concentration range that includes the IC₅₀ as determined in cell culture. Such information can be used to more accurately determine useful doses in humans. Levels in plasma may be measured, for example, by high performance liquid chromatography.

The PDE9 inhibitors used in accordance with the present invention may also be used in conjunction with other pharmaceutical agents for the treatment of the diseases, conditions and/or disorders described herein. Therefore, methods of treatment that include administering PDE9 inhibitors in combination with other pharmaceutical agents are also provided. Suitable pharmaceutical agents that may be used in combination with the compounds of the present invention include anti-obesity agents such as 3 adrenergic receptor agonists, apolipoprotein-B secretion/microsomal triglyceride transfer protein (apo-B/MTP) inhibitors, peptide YY₃₋₃₆ and analogs thereof, MCR-4 agonists, cholecystokinin-A (CCK-A) agonists, monoamine reuptake inhibitors (e.g., sibutramine), sympathomimetic agents, cannabinoid receptor antagonists (e.g., rimonabant (SR-141,716A)), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e., orlistat), anorectic agents (e.g., a bombesin agonist), Neuropeptide-Y antagonists, thyromimetic agents, dehydroepiandrosterones or analogs thereof, glucocorticoid receptor agonists or antagonists, orexin receptor antagonists, glucagon-like peptide-1 receptor agonists, ciliary neurotrophic factors (e.g., Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists, IIβ-hydroxy steroid dehydrogenase-1 inhibitors and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, pseudoephedrine, and peptide YY₃₋₃₆ (including analogs thereof). Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared, e.g., as described in U.S. Pat. No. 4,929,629; bromocriptine can be prepared, e.g., as described in U.S. Pat. Nos. 3,752,814 and 3,752,888; orlistat can be prepared, e.g., as described in U.S. Pat. Nos. 5,274,143; 5,420,305; 5,540,917; and 5,643,874; and PYY₃₋₃₆ (including analogs) can be prepared, e.g., as described in U.S. Patent Appl. Publication No. 2002/0141985, and WO 03/027637.

The skilled artisan will appreciate that certain factors may influence the dosage and timing required to effectively treat a mammal including, but not limited to, the severity of the disease or disorder, previous treatments, the general health and/or age of the mammal, and other diseases present. Moreover, treatment of a mammal with a therapeutically effective amount of a PDE9 inhibitor can include a single treatment or, preferably, can include a series of treatments.

### Genetically-Modified Non-Human Mammals and Cells

The genetically-modified, non-human mammals and genetically-modified animal cells, including human cells, of the invention are heterozygous or homozygous for a modification that disrupts the PDE9 gene. The animal cells may be derived by genetically engineering cells in culture, or, in the case of non-human mammalian cells, the cells may be isolated from genetically-modified, non-human mammals.

### Disruption of the PDE9 Gene

In order to create genetically-modified non-human mammals and mammal cells of the invention, the PDE9 gene locus may be disrupted using techniques for genetic modification known in the art, including chemical mutagenesis (Rinchik, Trends in Genetics 7:15-21, 1991, Russell, Environmental & Molecular Mutagenesis 23 (Suppl. 24): 23-29, 1994), irradiation (Russell, supra), transgenic expression of PDE9 gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55, 1994; Larsson et al., Nucleic Acids Research 22: 2242-48, 1994) and, as further discussed below, the disruption of the PDE9 gene by the insertion of a foreign nucleic acid sequence into the PDE9 gene locus. Preferably, the foreign sequence is inserted by homologous recombination or by the insertion of a viral vector. Most preferably, the method of PDE9 gene disruption to create the genetically modified non-human mammals and animal cells of the invention is homologous recombination and includes a deletion of a portion of the endogenous PDE9 gene sequence.

The integration of the foreign sequence disrupts the PDE9 gene through one or more of the following mechanisms: by interfering with the PDE9 gene transcription or translation process (e.g., by interfering with promoter recognition, or by introducing a transcription termination site or a translational stop codon into the PDE9 gene); or by distorting the PDE9 gene coding sequence such that it no longer encodes a PDE9 polypeptide with normal function (e.g., by inserting a foreign coding sequence into the PDE9 gene coding sequence, by introducing a frameshift mutation or amino acid(s) substitution, or, in the case of a double crossover event, by deleting a portion of the PDE9 gene coding sequence that is required for expression of a functional PDE9 protein).

To insert a foreign sequence into a PDE9 gene locus in the genome of a cell to create the genetically modified non-human mammals and animal cells of the invention based upon the present description, the foreign DNA sequence is introduced into the cell according to a standard method known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Potter et al., Proc. Natl. Acad. Sci USA 81: 7161-65, 1984; Chu et al., Nucleic Acids Res. 15: 1311-26, 1987; Thomas and Capecchi, Cell 51: 503-12, 1987; Baum et al., Biotechniques 17: 1058-62, 1994; Biewenga et al., J. Neuroscience Methods 71: 67-75, 1997; Zhang et al., Biotechniques 15: 868-72, 1993; Ray and Gage, Biotechniques 13: 598-603, 1992; Lo, Mol. Cell. Biol. 3: 1803-14, 1983; Nickoloff et al., Mol. Biotech. 10: 93-101, 1998; Linney et al., Dev. Biol. (Orlando) 213: 207-16, 1999; Zimmer and Gruss, Nature 338: 150-153, 1989; and Robertson et al., Nature 323: 445-48, 1986). The preferred method for introducing foreign DNA into a cell is electroporation.

### Homologous Recombination

The method of homologous recombination targets the PDE9 gene for disruption by introducing a PDE9 gene targeting vector into a cell containing a PDE9 gene. The ability of the vector to target the PDE9 gene for disruption stems from using a nucleotide sequence in the vector that is homologous, i.e., related, to the PDE9 gene. This homology region facilitates hybridization between the vector and the endogenous sequence of the PDE9 gene. Upon hybridization, the probability of a crossover event between the targeting vector and genomic sequences greatly increases. This crossover event results in the integration of the vector sequence into the PDE9 gene locus and the functional disruption of the PDE9 gene.

General principles regarding the construction of vectors used for targeting are reviewed in Bradley et al. (Biotechnol. 10: 534, 1992). Two different types of vector can be used to insert DNA by homologous recombination: an insertion vector or a replacement vector. An insertion vector is circular DNA which contains a region of PDE9 gene homology with a double stranded break. Following hybridization between the homology region and the endogenous PDE9 gene, a single crossover event at the double stranded break results in the insertion of the entire vector sequence into the endogenous gene at the site of crossover.

The more preferred vector to create the genetically modified non-human mammals and animals cells of the invention by homologous recombination is a replacement vector, which is colinear rather than circular. Replacement vector integration into the PDE9 gene requires a double crossover event, i.e., crossing over at two sites of hybridization between the targeting vector and the PDE9 gene. This double crossover event results in the integration of a vector sequence that is sandwiched between the two sites of crossover into the PDE9 gene and the deletion of the corresponding endogenous PDE9 gene sequence that originally spanned between the two sites of crossover (see, e.g., Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector used to create the genetically modified non-human mammals and animal cells of the invention is generally at least 100 nucleotides in length. Most preferably, the homology region is at least 1-5 kilobases (kb) in length. Although there is no demonstrated minimum length or minimum degree of relatedness required for a homology region, targeting efficiency for homologous recombination generally corresponds with the length and the degree of relatedness between the targeting vector and the PDE9 gene locus. In the case where a replacement vector is used, and a portion of the endogenous PDE9 gene is deleted upon homologous recombination, an additional consideration is the size of the deleted portion of the endogenous PDE9 gene. If this portion of the endogenous PDE9 gene is greater than 1 kb in length, then a targeting cassette with regions of homology that are longer than 1 kb is recommended to enhance the efficiency of recombination. Further guidance regarding the selection and use of sequences effective for homologous recombination, based on the present description, is described in the literature (see, e.g., Deng and Capecchi, Mol. Cell. Biol. 12: 3365-3371, 1992; Bollag et al., Annu. Rev. Genet. 23: 199-225, 1989; and Waldman and Liskay, Mol. Cell. Biol. 8: 5350-5357, 1988).

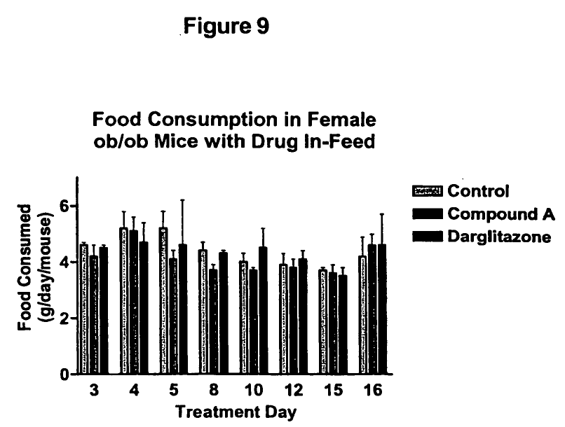

As those skilled in the art will recognize based upon the present invention, a wide variety of cloning vectors may be used as vector backbones in the construction of the PDE9 gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79, phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, FIG. 9. 16.1).

The specific host employed for propagating the targeting vectors of the present invention is not critical. Examples include E. coli K12 RR1 (Bolivar et al., Gene 2: 95, 1977), E. coli K12 HB101 (ATCC No. 33694), E. coli MM21 (ATCC No. 336780), E. coli DH1 (ATCC No. 33849), E. coli strain DH5α, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

To create the targeting vector, a PDE9 gene targeting construct is added to an above-described vector backbone. The PDE9 gene targeting constructs of the invention have at least one PDE9 gene homology region. To make the PDE9 gene homology regions, a PDE9 genomic or cDNA sequence is used as a basis for producing PCR primers. These primers are used to amplify the desired region of the PDE9 sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for PDE9 gene disruption. a PDE9 cDNA sequence can be used in making a PDE9 targeting vector (e.g., GenBank® NM008804 (murine) or GenBank® NM002606 (human)).

Preferably, the targeting constructs of the invention also include an exogenous nucleotide sequence encoding a positive marker protein. The stable expression of a positive marker after vector integration confers an identifiable characteristic on the cell, ideally, without compromising cell viability. Therefore, in the case of a replacement vector, the marker gene is positioned between two flanking homology regions so that it integrates into the PDE9 gene following the double crossover event in a manner such that the marker gene is positioned for expression after integration.

It is preferred that the positive marker protein is a selectable protein; the stable expression of such a protein in a cell confers a selectable phenotypic characteristic, i.e., the characteristic enhances the survival of the cell under otherwise lethal conditions. Thus, by imposing the selectable condition, one can isolate cells that stably express the positive selectable marker-encoding vector sequence from other cells that have not successfully integrated the vector sequence on the basis of viability. Examples of positive selectable marker proteins (and their agents of selection) include neo (G418 or kanomycin), hyg (hygromycin), hisD (histidinol), gpt (xanthine), ble (bleomycin), and hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or green fluorescent protein (see, e.g., Current Protocols in Cytometry, Unit 9.5, and Current Protocols in Molecular Biology, Unit 9.6, John Wiley & Sons, New York, N.Y., 2000).

The above-described positive selection step does not distinguish between cells that have integrated the vector by targeted homologous recombination at the PDE9 gene locus versus random, non-homologous integration of vector sequence into any chromosomal position. Therefore, when using a replacement vector for homologous recombination to make the genetically modified non-human mammals and animal cells of the invention, it is also preferred to include a nucleotide sequence encoding a negative selectable marker protein. Expression of a negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2-deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the PDE9 gene and the two regions of homology in the targeting construct excludes the sequence encoding the negative selectable marker from integration. Thus, by imposing the negative condition, cells that have integrated the targeting vector by random, non-homologous recombination lose viability.

The above-described combination of positive and negative selectable markers is preferred in a targeting construct used to make the genetically modified non-human mammals and animal cells of the invention because a series of positive and negative selection steps can be designed to more efficiently select only those cells that have undergone vector integration by homologous recombination, and, therefore, have a potentially disrupted PDE9 gene. Further examples of positive-negative selection schemes, selectable markers, and targeting constructs are described, for example, in U.S. Pat. No. 5,464,764, WO 94/06908, U.S. Pat. No. 5,859,312, and Valancius and Smithies, Mol. Cell. Biol. 11:1402, 1991.

For a marker protein to be stably expressed upon vector integration, the targeting vector may be designed so that the marker coding sequence is operably linked to the endogenous PDE9 gene promoter upon vector integration. Expression of the marker is then driven by the PDE9 gene promoter in cells that normally express the PDE9 gene. Alternatively, each marker in the targeting construct of the vector may contain its own promoter that drives expression independent of the PDE9 gene promoter. This latter scheme has the advantage of allowing for expression of markers in cells that do not typically express the PDE9 gene (Smith and Berg, Cold Spring Harbor Symp. Quant. Biol. 49: 171, 1984; Sedivy and Sharp, Proc. Natl. Acad. Sci. (USA) 86: 227, 1989; Thomas and Capecchi, Cell 51: 503, 1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see generally, Sambrook et al., Molecular Cloning, Vols. I-III, Cold Spring Harbor Laboratory Press, Cold Spring Harbor, N.Y., 1989, and Current Protocols in Molecular Biology, John Wiley & Sons, New York, N.Y., 2000; Stratagene, La Jolla, Calif.).

To confirm whether cells have integrated the vector sequence into the targeted PDE9 gene locus while making the genetically modified non-human mammals and animal cells of the invention, primers or genomic probes that are specific for the desired vector integration event can be used in combination with polymerase chain reaction (PCR) or Southern blot analysis to identify the presence of the desired vector integration into the PDE9 gene locus (Erlich et al., Science 252: 1643-51, 1991; Zimmer and Gruss, Nature 338: 150, 1989; Mouellic et al., Proc. Natl. Acad. Sci. (USA) 87: 4712, 1990; and Shesely et al., Proc. Natl. Acad. Sci. (USA) 88: 4294, 1991).

### Gene Trapping

Another method available for inserting a foreign nucleic acid sequence into the PDE9 gene locus to disrupt the PDE9 gene, based on the present description, is gene trapping. This method takes advantage of the cellular machinery present in all mammalian cells that splices exons into mRNA to insert a gene trap vector coding sequence into a gene in a random fashion. Once inserted, the gene trap vector creates a mutation that may disrupt the trapped PDE9 gene. In contrast to homologous recombination, this system for mutagenesis creates largely random mutations. Thus, to obtain a genetically-modified cell that contains a disrupted PDE9 gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91, 1992; Brenner et al., Proc. Nat. Acad. Sci. USA 86: 5517-21, 1989; Chang et al., Virology 193: 737-47, 1993; Friedrich and Soriano, Methods Enzymol. 225: 681-701, 1993; Friedrich and Soriano, Genes Dev. 5: 1513-23, 1991; Goff, Methods Enzymol. 152: 469-81, 1987; Gossler et al., Science 244: 463-65, 1989; Hope, Develop. 113: 399-408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918, 1992; von Melchner and Ruley, J. Virol. 63: 3227-3233, 1989; and Yoshida et al., Transgen. Res. 4: 277-87, 1995).

Individual mutant cell lines containing a disrupted PDE9 gene are identified in a population of mutated cells using, for example, reverse transcription (RT) and PCR to identify a mutation in a PDE9 gene sequence. This process can be streamlined by pooling clones. For example, to find an individual clone containing a disrupted PDE9 gene, RT-PCR is performed using one primer anchored in the gene trap vector and the other primer located in the PDE9 gene sequence. A positive RT-PCR result indicates that the vector sequence is encoded in the PDE9 gene transcript, indicating that the PDE9 gene has been disrupted by a gene trap integration event (see, e.g., Sands et al., WO 98/14614, U.S. Pat. No. 6,080,576).

### Temporal, Spatial, and Inducible PDE9 Gene Disruptions

In certain embodiments of the present invention, a functional disruption of the endogenous PDE9 gene occurs at specific developmental or cell cycle stages (temporal disruption) or in specific cell types (spatial disruption). In other embodiments, the PDE9 gene disruption is inducible when certain conditions are present. A recombinase excision system, such as a Cre-Lox system, may be used to activate or inactivate the PDE9 gene at a specific developmental stage, in a particular tissue or cell type, or under particular environmental conditions. Generally, methods utilizing Cre-Lox technology are carried out as described by Torres and Kuhn, Laboratory Protocols for Conditional Gene Targeting, Oxford University Press, 1997. Methodology similar to that described for the Cre-Lox system can also be employed utilizing the FLP-FRT system. Further guidance regarding the use of recombinase excision systems for conditionally disrupting genes by homologous recombination or viral insertion is provided, for example, in U.S. Pat. No. 5,626,159; U.S. Pat. No. 5,527,695; U.S. Pat. No. 5,434,066; WO 98/29533; U.S. Pat. No. 6,228,639; Orban et al., Proc. Nat. Acad. Sci. USA 89: 6861-65, 1992; O'Gorman et al., Science 251: 1351-55, 1991; Sauer et al., Nucleic Acids Research 17: 147-61, 1989; Barinaga, Science 265: 26-28, 1994; and Akagi et al., Nucleic Acids Res. 25: 1766-73, 1997. More than one recombinase system can be used to genetically modify a non-human mammal or animal cell of the present invention.

When using homologous recombination to disrupt the PDE9 gene in a temporal, spatial, or inducible fashion, using a recombinase system such as the Cre-Lox system, a portion of the PDE9 gene coding region is replaced by a targeting construct comprising the PDE9 gene coding region flanked by loxP sites. Non-human mammals and animal cells carrying this genetic modification contain a functional, loxP-flanked PDE9 gene. The temporal, spatial, or inducible aspect of the PDE9 gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched PDE9 gene coding sequence, resulting in a functional disruption of the PDE9 gene (Rajewski et al., J. Clin. Invest. 98: 600-03, 1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94: 14559-63, 1997; Feil et al., Proc. Natl. Acad. Sci. USA 93: 10887-90, 1996; and Kuhn et al., Science 269: 1427-29, 1995).

A cell containing both a Cre recombinase transgene and loxP-flanked PDE9 gene can be generated through standard transgenic techniques or, in the case of genetically-modified, non-human mammals, by crossing genetically-modified, non-human mammals wherein one parent contains a loxP flanked PDE9 gene and the other contains a Cre recombinase transgene under the control of the desired promoter. Further guidance regarding the use of recombinase systems and specific promoters to temporally, spatially, or conditionally disrupt the PDE9 gene is found, for example, in Sauer, Meth. Enz. 225: 890-900, 1993; Gu et al., Science 265: 103-06, 1994; Araki et al., J. Biochem. 122: 977-82, 1997; Dymecki, Proc. Natl. Acad. Sci. 93: 6191-96, 1996; and Meyers et al., Nature Genetics 18: 136-41, 1998.

An inducible disruption of the PDE9 gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous PDE9 gene regulatory element and a transgene expressing a tetracycline-controllable repressor (TetR). In such a cell, the administration of tetracycline activates the TetR which, in turn, inhibits PDE9 gene expression and, therefore, disrupts the PDE9 gene (St.-Onge et al., Nucleic Acids Res. 24: 3875-77, 1996; U.S. Pat. No. 5,922,927).

The above-described systems for temporal, spatial, and inducible disruptions of the PDE9 gene can also be adopted when using gene trapping as the method of genetic modification, for example, as described, in WO 98/29533 and U.S. Pat. No. 6,288,639, for creating the genetically modified non-human mammals and animal cells of the invention.

Creating Genetically-Modified. Non-Human Mammals and Animal Cells

The above-described methods for genetic modification can be used to disrupt a PDE9 gene in virtually any type of somatic or stem cell derived from an animal to create the genetically modified animal cells of the invention. Genetically-modified animal cells of the invention include, but are not limited to, mammalian cells, including human cells, and avian cells. These cells may be derived from genetically engineering any animal cell line, such as culture-adapted, tumorigenic, or transformed cell lines, differentiated genetically-engineered ES cells, or they may be isolated from a genetically-modified, non-human mammal carrying the desired PDE9 genetic modification.

The cells may be heterozygous or homozygous for the disrupted PDE9 gene. To obtain cells that are homozygous for the PDE9 gene disruption (−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second PDE9 gene allele (Abuin and Bradley, Mol. Cell. Biol. 16: 1851-56, 1996; Sedivy et al., T. I. G. 15: 88-90, 1999; Cruz et al., Proc. Natl. Acad. Sci. (USA) 88: 7170-74, 1991; Mortensen et al., Proc. Natl. Acad. Sci. (USA) 88: 703640, 1991; te Riele et al., Nature (London) 348: 649-651, 1990).

An alternative strategy for obtaining ES cells that are PDE9−/− is the homogenotization of cells from a population of cells that is heterozygous for the PDE9 gene disruption (PDE9+/−). The method uses a scheme in which PDE9+/− targeted clones that express a selectable drug resistance marker are selected against a very high drug concentration; this selection favors cells that express two copies of the sequence encoding the drug resistance marker and are, therefore, homozygous for the PDE9 gene disruption (Mortensen et al., Mol. Cell. Biol. 12: 2391-95, 1992). In addition, genetically-modified animal cells can be obtained from genetically-modified PDE9−/− non-human mammals that are created by mating non-human mammals that are PDE9+/− in germline cells, as further discussed below.

Following the genetic modification of the desired cell or cell line, the PDE9 gene locus can be confirmed as the site of modification by PCR analysis according to standard PCR or Southern blotting methods known in the art (see, e.g., U.S. Pat. No. 4,683,202; and Erlich et al., Science 252: 1643, 1991). Further verification of the functional disruption of the PDE9 gene may also be made if PDE9 gene messenger RNA (mRNA) levels and/or PDE9 polypeptide levels are reduced in cells that normally express the PDE9 gene. Measures of PDE9 gene mRNA levels may be obtained by using RT-PCR, Northern blot analysis, or in situ hybridization. The quantification of PDE9 polypeptide levels produced by the cells can be made, for example, by standard immunoassay methods known in the art. Such immunoassays include, but are not limited to, competitive and non-competitive assay systems using techniques such as RIAs (radioimmunoassays), ELISAs (enzyme-linked immunosorbent assays), “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

Preferred genetically-modified animal cells of the invention are embryonic stem (ES) cells and ES-like cells. These cells are derived from the preimplantation embryos and blastocysts of various species, such as mice (Evans et al., Nature 129:154-156, 1981; Martin, Proc. Natl. Acad. Sci., USA, 78: 7634-7638, 1981), pigs and sheep (Notanianni et al., J. Reprod. Fert. Suppl., 43: 255-260, 1991; Campbell et al., Nature 380: 64-68, 1996) and primates, including humans (Thomson et al., U.S. Pat. No. 5,843,780, Thomson et al., Science 282: 1145-1147, 1995; and Thomson et al., Proc. Natl. Acad. Sci. USA 92: 7844-7848, 1995). Success at homologous recombination-mediated gene disruption in human ES cells has been reported (Zwaka and Thomson, Nature Biotech. 21: 319-21, 2003).

These types of cells are pluripotent, that is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995; Li et al., Curr. Biol. 8: 971, 1998; Klug et al., J. Clin. Invest. 98: 216-24, 1996; Lieschke et al., Exp. Hematol. 23: 328-34, 1995; Yamane et al., Blood 90: 3516-23, 1997; and Hirashima et al., Blood 93: 1253-63, 1999.

The particular embryonic stem cell line that is used for genetic modification is not critical; exemplary murine ES cell lines include AB-1 (McMahon and Bradley, Cell 62:1073-85, 1990), E14 (Hooper et al., Nature 326: 292-95, 1987), D3 (Doetschman et al., J. Embryol. Exp. Morph. 87: 27-45, 1985), CCE (Robertson et al, Nature 323: 445-48, 1986), RW4 (Genome Systems, St. Louis, Mo.), and DBA/1 lacJ (Roach et al., Exp. Cell Res. 221: 520-25, 1995); an exemplary human ES cell line is H1.1 cells (Zwaka and Thomson, Nature Biotech. 21: 319-321, 2003). Genetically-modified murine ES cells may be used to generate genetically-modified mice, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

Following confirmation that the ES cells contain the desired functional disruption of the PDE9 gene, these ES cells are then injected into suitable blastocyst hosts for generation of chimeric mice according to methods known in the art (Capecchi, Trends Genet. 5: 70, 1989). The particular mouse blastocysts employed in the present invention are not critical. Examples of such blastocysts include those derived from C57BL6 mice, C57BL6 Albino mice, Swiss outbred mice, CFLP mice, and MFI mice. Alternatively ES cells may be sandwiched between tetraploid embryos in aggregation wells (Nagy et al., Proc. Natl. Acad. Sci. USA 90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory press, Cold Spring Harbor, N.Y. 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the PDE9 gene disruption. Generally, such offspring contain some cells that are derived from the genetically-modified donor ES cell as well as other cells derived from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells.

The mating of chimeric mice that contain the PDE9 gene disruption in germ line cells produces progeny that possess the PDE9 gene disruption in all germ line cells and somatic cells. Mice that are heterozygous for the PDE9 gene disruption can then be crossed to produce homozygotes (see, e.g., U.S. Pat. No. 5,557,032; and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the PDE9 gene. When inserting a DNA vector into a somatic cell to mutate the PDE9 gene, it is preferred that a promoterless marker be used in the vector such that vector integration into the PDE9 gene results in expression of the marker under the control of the PDE9 gene promoter (Sedivy and Dutriaux, T. I. G. 15: 88-90, 1999; McCreath et al., Nature 29: 1066-69, 2000). Nuclei from donor cells which have the appropriate PDE9 gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The present invention also encompasses the progeny of the genetically-modified, non-human mammals and genetically-modified animal cells. While the progeny are heterozygous or homozygous for the genetic modification that disrupts the PDE9 gene, they may not be genetically identical to the parent non-human mammals and animal cells due to mutations or environmental influences, besides that of the original genetic disruption of the PDE9 gene, that may occur in succeeding generations.

The cells from a non-human genetically modified animal can be isolated from tissue or organs using techniques known to those of skill in the art. In one embodiment, the genetically modified cells of the invention are immortalized. In accordance with this embodiment, cells can be immortalized by genetically engineering the telomerase gene, an oncogene, e.g., mos or v-src, or an apoptosis-inhibiting gene, e.g., bcl-2, into the cells. Alternatively, cells can be immortalized by fusion with a hybridization partner utilizing techniques known to one of skill in the art.

### “Humanized” Non-Human Mammals and Animal Cells

The genetically-modified non-human mammals and animal cells (non-human) of the invention containing a disrupted endogenous PDE9 gene can be further modified to express the human PDE9 sequence (referred to herein as “humanized”). A preferred method for humanizing cells involves replacing the endogenous PDE9 sequence with nucleic acid sequence encoding the human PDE9 sequence (Jakobsson et al., Proc. Natl. Acad. Sci. USA 96: 7220-25, 1999) by homologous recombination. The vectors are similar to those traditionally used as targeting vectors with respect to the 5′ and 3′ homology arms and positive/negative selection schemes. However, the vectors also include sequence that, after recombination, either substitutes the human PDE9 coding sequence for the endogenous sequence, or effects base pair changes, exon substitutions, or codon substitutions that modify the endogenous sequence to encode the human PDE9. Once homologous recombinants have been identified, it is possible to excise any selection-based sequences (e.g., neo) by using Cre or Flp-mediated site directed recombination (Dymecki, Proc. Natl. Acad. Sci. 93: 6191-96, 1996).

When substituting the human PDE9 sequence for the endogenous sequence, it is preferred that these changes are introduced directly downstream of the endogenous translation start site. This positioning preserves the endogenous temporal and spatial expression patterns of the PDE9 gene. The human sequence can be the full length human cDNA sequence with a polyA tail attached at the 3′ end for proper processing or the whole genomic sequence (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-17980, 1997, Reaume et al., J. Biol. Chem. 271: 23380-23388, 1996, and Scott et al., U.S. Pat. No. 5,777,194).

Another method for creating such “humanized” organisms is a two step process involving the disruption of the endogenous gene followed by the introduction of a transgene encoding the human sequence by pronuclear microinjection into the knock-out embryos.

### Uses for the Genetically-Modified Non-Human Mammals and Animal Cells

PDE9 function and therapeutic relevance can be further elucidated by additional investigation into the phenotype of PDE9−/− non-human mammals and animals cells of the invention, as illustrated, for example, in the Examples hereinbelow. For example, the genetically-modified PDE9−/− non-human mammals and animal cells can be used to determine whether the PDE9 plays a role in causing or preventing symptoms or phenotypes to develop in certain models of disease, e.g., obesity, eating disorders, cardiovascular disorders, insulin resistance syndrome, hypertension, and/or type 2 diabetes. If a symptom or phenotype is different in a PDE9−/− non-human mammal or animal cell as compared to a wild type (PDE9+/+) or PDE9+/− non-human mammal or animal cell, then the PDE9 polypeptide plays a role in regulating functions associated with the symptom or phenotype. Examples of testing that can be used to assess PDE9 function include comparing PDE9−/− mice to wild type mice in terms of body weight, body fat, blood pressure, glucose/insulin metabolism (e.g., glucose uptake in isolated tissues, alterations in the activity of glycogen metabolism enzymes, alterations in glycogen levels in liver or muscle, and/or alterations in body composition), and changes in the activity or phosphorylation state of components in the insulin signaling pathway.

In addition, under circumstances in which an agent has been identified as a PDE9 agonist or antagonist (e.g., the agent significantly modifies one or more of the PDE9 polypeptide activities when the agent is administered to a PDE9+/+ or PDE9+/− non-human mammal or animal cell), the genetically-modified PDE9−/− non-human mammals and animal cells of the invention are useful to characterize any other effects caused by the agent besides those known to result from the (ant)agonism of PDE9 (i.e., the non-human mammals and animal cells can be used as negative controls). For example, if the administration of the agent causes an effect in a PDE9+/+ non-human mammal or animal cell that is not known to be associated with PDE9 polypeptide activity, then one can determine whether the agent exerts this effect solely or primarily through modulation of PDE9 by administering the agent to a corresponding PDE9−/− non-human mammal or animal cell. If this effect is absent, or is significantly reduced, in the PDE9−/− non-human mammal or animal cell, then the effect is mediated, at least in part, by PDE9. However, if the PDE9−/− non-human mammal or animal cell exhibits the effect to a degree comparable to the PDE9+/+ or PDE9+/− non-human mammal or animal cell, then the effect is mediated by a pathway that does not involve PDE9 signaling.

Furthermore, if an agent is suspected of possibly exerting an effect predominantly via a PDE9 pathway, then the PDE9−/− non-human mammals and animal cells are useful as negative controls to test this hypothesis. If the agent is indeed acting through PDE9, then the PDE9−/− non-human mammals and animal cells, upon administration of the agent, should not demonstrate the same effect observed in the PDE9+/+ non-human mammals or animal cells.

The genetically modified non-human mammals and animal cells of the invention can also be used to identify genes whose expression is differentially regulated in PDE9+/− or PDE9−/− non-human mammals or animal cells relative to their respective wild type control. Techniques known to those of skill in the art can be used to identify such genes based upon the present description. For example, DNA arrays can be used to identify genes whose expression is differentially regulated in PDE9+/− or PDE9−/− mice to compensate for a deficiency in PDE9 expression. DNA arrays are known to those of skill in the art (see, e.g., Aigner et al., Arthritis and Rheumatism 44: 2777-89, 2001; U.S. Pat. No. 5,965,352; Schena et al., Science 270: 467-470, 1995; Schena et al., Proc. Natl. Acad. Sci. USA 93: 10614-10619, 1996; DeRisi et al., Nature Genetics 14: 457-460, 1996; Shalon et al., Genome Res. 6: 639-645, 1996; and Schena et al., Proc. Natl. Acad. Sci. (USA) 93:10539-11286, 1995; U.S. Pat. No. 5,474,796; U.S. Pat. No. 5,605,662; WO 95/25116; WO 95/35505; Heller et al., Proc. Natl. Acad. Sci. 94: 2150-2155, 1997).

A chemical coupling procedure and an ink jet device may be used to synthesize array elements on the surface of a substrate. An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, expressed sequence tags (ESTs), or fragments thereof may comprise the elements of a microarray. Fragments suitable for hybridization may be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., ultra-violet cross-linking followed by thermal and chemical treatments and subsequent drying. Fluorescent probes are prepared and used for hybridization to the elements on the substrate. The substrate is analyzed by procedures well known in the art, for example, by scanning and analyzing images of a microarray.

In addition, the genetically modified non-human mammals and animal cells of the invention can also be used to identify proteins whose expression profile or postranslational modification is altered in PDE9+/− or PDE9−/− non-human mammals or animal cells relative to their respective wild type control. Techniques known to those of skill in the art can be used to identify such proteins based upon the present description. For example, proteomic assays can be used to identify proteins whose expression profile or postranslational modification is altered in PDE9+/− or PDE9−/− mice to compensate for a deficiency in PDE9 expression. Proteomic assays are known to those of skill in the art (see, e.g., Conrads et al., Biochem. Biophys. Res. Commun. 290: 896-890, 2002; Dongre et al., Biopolymers 60: 206-211, 2001; Van Eyk, Curr Opin Mol Ther 3: 546-553, 2001; Cole et al., Electrophoresis 21: 1772-1781, 2000; Araki et al., Electrophoresis 21: 180-1889, 2000).

### EXAMPLES

### 1. Preparation of PDE9 Targeting Vector

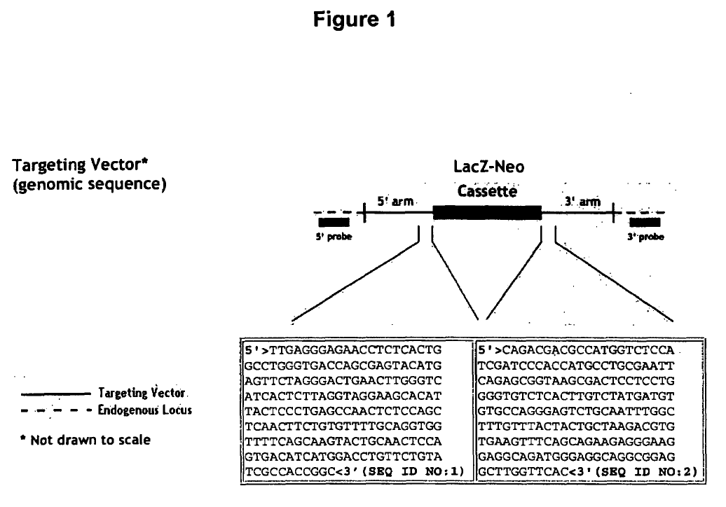

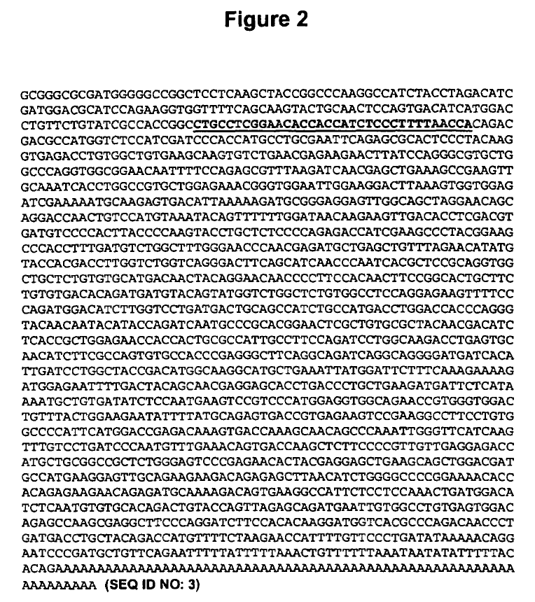

A targeting vector construct was designed according to the scheme shown in FIG. 1. The construct contained two arms homologous to the murine PDE9 genomic sequence: a 0.9 kb 5′ homology arm and a 4.3 kb 3′ homology arm. These arms sandwiched a LacZ-Neo construct. DNA containing the targeting construct was inserted into ES R1 cells by electroporation (Deng et al., Dev. Biol. 185: 42-54, 1997; Udy et al., Exp. Cell Res. 231: 296-301, 1997). Upon homologous recombination, base pairs 142-175 of the PDE9 cDNA coding sequence shown in FIG. 2 (base pairs 142-175 are underlined) were deleted from the endogenous gene and replaced by the LacZ Neo cassette. ES cells that were neomycin resistant were analyzed by Southern blot to confirm disruption of a PDE9 gene. These targeted ES cells were then used for generation of chimeric mice by injecting the cells into blastocysts and implanting the blastocysts into pseudopregnant female mice (Capecchi et al., Trends Genet. 5: 70, 1989, Hogan et al., Manipulating the Mouse Embryo: A Laboratory Manual, Cold Spring Harbor Laboratory, 1988; and Teratocarcinomas and Embryonic Stem Cells: A Practical Approach, E. J. Robertson, ed., IRL Press, Washington, D.C., 1987). Chimeric mice were then bred with C57BL/6 (Jackson Laboratories, Bar Harbor, Me.) mice to create F1 PDE9+/− heterozygotes, which were in turn bred to produce F2 PDE9−/− homozygous mice (Charles River Laboratories, Wilmington, Mass.). The functional disruption of the PDE9 gene in the heterozygotes and homozygotes was confirmed by PCR and Southern blot analysis.

### 2. Effect of PDE9 Gene Disruption on Body Weight, Body Composition, and Metabolites

### Methods

Male and female PDE9 KO (−/−) mice, as previously described, and wild type (+/+) littermate controls were allowed to acclimate for at least one week prior to the start of the study and were given free access to water and D11 mouse chow (Purina, Brentwood, Mo.).

Male and female mice (aged 17-19 weeks) were divided into four experimental groups with two to five mice per cage. One group of control mice of each gender remained on D11 mouse chow and the remaining groups of each gender were switched to a diet composed of 58 kcal % fat (D12331 Rodent Diet, Research Diets, Inc., New Brunswick, N.J.) for the duration of the 6 week study. Body weight was determined on Day 0 and monitored weekly. Adipose depot mass was analyzed on Day 0 and at the end of the study, as further described below.

On Day 1, plasma glucose was determined via retro-orbital blood samples. 25 μL of blood was added to 100 μL of 0.025 percent heparinized-saline in microtubes (Denville Scientific, Inc., Metuchen, N.J.). The tubes were spun at the highest setting in a Beckman Microfuge 12 for 2 minutes. Plasma was collected for plasma glucose and triglyceride determination, as further described below. During the course of the study, body weight and food consumption were assessed, and blood samples were taken at approximately 8 am for plasma glucose and triglyceride measures, as further described below.

On the morning of the last day of the study, blood samples were taken via retro-orbital sinus for plasma glucose and triglyceride determination. The mice were then sacrificed and about one milliliter of blood was collected in Microtainer® plasma separator tubes with lithium heparin (Becton-Dickinson, Inc., Franklin Lakes, N.J.). The tubes were spun in a Beckman Microfuge 12 at the maximum setting for five minutes. Plasma was collected in 1.5 ml Eppendorf tubes, snap frozen in liquid nitrogen, and stored at −80° C. until analyzed for insulin, fructosamine, or cGMP levels.

Plasma glucose, triglycerides, and fructosamine were measured using the Roche/Hitachi 912 Clinical Chemistry Analyzer (Roche Diagnostics Corp., Indianapolis, Ind.). Plasma cGMP was measured using the BioTrak™ enzyme-immunoassay system (Amersham, Piscataway, N.J.). Plasma insulin was assessed via a similar technique using the Mercodia ELISA Insulin kit supplied by ALPCO (Uppsala, Sweden). All assays were conducted according to each manufacturer's instructions.

Quantification of adipose depot mass was done five days prior to the end of the study. To assess the adipose depot mass, 3600 radioscopic images of the mice were obtained using a commercially available micro computed tomography (CT) system (MicroCAT®, ImTek Inc., Oak Ridge, Tenn.) with a high-resolution CCD/phosphor screen detector. The scanner consisted of a cylindrical diameter/long field view of 36 mm/36 mm with a spatial resolution of less than 50 μM. The X-ray source was biased at 40 KeV with the anode current set to 0.4 mA. Anesthetized mice were placed on a radiotransparent mouse bed in an anatomically correct supine position, caudal end closest to the micro CT with the rostral end held in place against an anesthesia delivery tube. An initial radiographic image was acquired at 90° to the plane of the mouse bed to allow correct positioning of the mouse by centering the scan acquisition area at the level of the iliac crest of each mouse. Once correct alignment was assured, each animal was scanned. Each scan consisted of 196 individual projections with an exposure time of 250 μs/projection; total image acquisition time was approximately 12 minutes at 145 μM resolution.

Image reconstruction, whereby the 196 projections acquired in the micro CT scan of the mouse were manipulated to produce two-dimensional cross sectional images of the mouse, was performed using the MicroCAT® Reconstruction, Visualization, and Analysis Software (ImTek Inc., Oak Ridge, Tenn.) (Paulus et al., Neoplasia 2: 62-70, 2000). Two sets of reconstructed images per scan were generated for each mouse for the determination of individual fat depot mass. The first set of six reconstructed images provided a montage for the analysis of inguinal and epididymal adipose tissue depot mass. The second reconstruction set consisted of nine slices, determined by both intervertebral and midvertebral landmarks, and was used to determine retroperitoneal and mesenteric adipose tissue depot mass.

For image analysis, reconstructed bitmap images were converted to TIFF images. The TIFF images were subsequently analyzed and fat depot mass determined using Scion Image for Windows® (Scion Corporation, Frederick Md.). Demarcation lines separating individual fat depots were placed using the paintbrush tool (pixel size #3) and total pixel counts of each adipose region determined by the Scion Image software. An upper and lower pixel intensity threshold was chosen, in this study, a look-up-table (LUT) of between 115-187 was determined to be optimal for capturing the adipose depot.

Average pixel number between each slice was calculated (sliceₙ+sliceₙ₊₁)/2). Total pixel number, representing the individual fat depots, was calculated by multiplying the average pixel number between each slice by the average pixel number of each slice. Finally, the pixel count was converted into depot mass with the following equation: Depot mass (mg)=0.000915 g/ul×0.000757 μl/voxel×1000 mg/g×voxel count. The first factor corrects for specific gravity of glyceryl trioleate, representative of the density of the primary storage form of lipid in adipose tissue, i.e., triglyceride. The second factor is the volume per pixel and the third factor converts the resulting mass into mg units.

Results

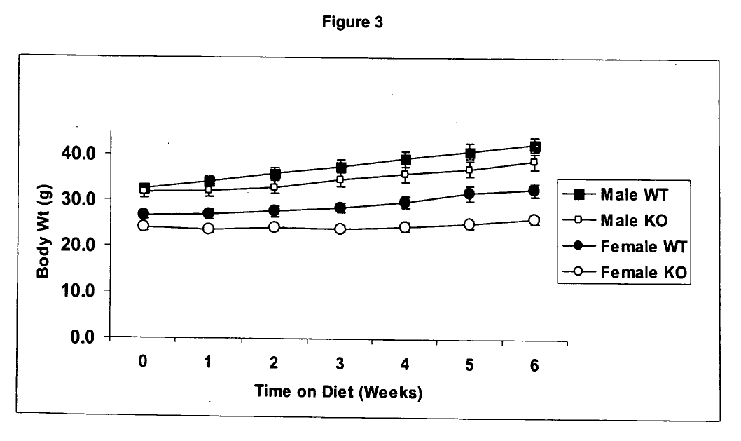

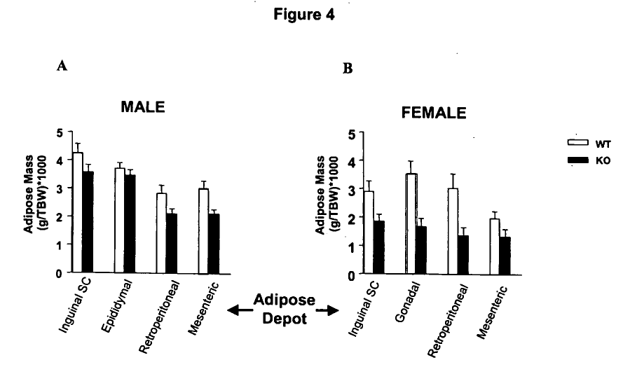

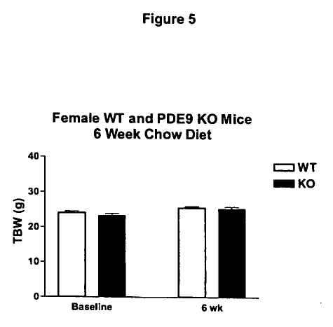

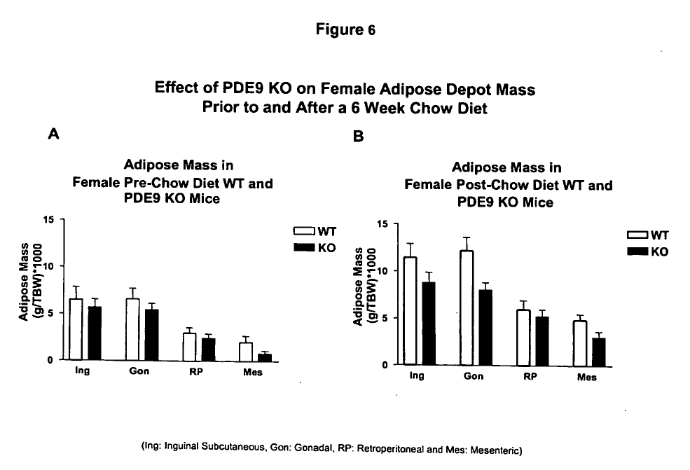

PDE9 disruption resulted in a decreased body weight gain and reduced body weight while on a high fat diet in both male and female KO mice as compared to their wild type counterparts (FIG. 3). Female mice also demonstrated a 6% decrease in body length. In conjunction with the decreased body weight, the male and female KO mice also demonstrated decreased fat mass in various adipose depots (FIG. 4). In male KO mice, significant decreases were seen in the retroperitoneal and mesenteric adipose depots; in female KO mice, significant decreases were seen in the inguinal, gonadal, and retroperitoneal depots. By comparison, in female mice fed a standard chow diet, no differences in body weight were observed between KO and wild type mice (FIG. 5) and the trend towards decreased adipose fat mass was significant in only the gonadal adipose depot (FIG. 6).

With respect to plasma metabolites following the high fat diet, female KO mice demonstrated increased cGMP, decreased glucose, and decreased insulin (Table 1); male KO mice demonstrated a trend towards increased cGMP and a trend toward decreased glucose (Table 1).

TABLE 1
Plasma Metabolites Following 6-week High-fat Diet

| | Female | | | Male | | |
|---|---|---|---|---|---|---|
| | WT | KO | p value | WT | KO | p value |
| cGMP | 8.0± 0.50 | 13.7 ± 0.6 | <0.01 | 8.2 ± 1.0 | 11.5 ± 1.8 | 0.14 |
| pmol/ml | | | | | | |
| Glucose | 175± 8 | 152 ± 6 | 0.03 | 179 ± 78 | 164 ± 8 | 0.21 |
| mg % | | | | | | |
| Insulin | 1.58± 0.25 | 0.88 ± 0.15 | 0.03 | 3.53 ± 0.48 | 3.28 ± 0.48 | 0.72 |
| ng/ml | | | | | | |
| Triglycerides | 111± 6 | 119 ± 9 | 0.42 | 200 ± 13 | 191 ± 1 | 0.61 |
| mg % | | | | | | |

### 3. Effect of Pharmacologic PDE9 Inhibition on Body Weight, Body Composition, and Food Intake in ob/ob Mice

Methods

Female ob/ob mice obtained from Jackson Laboratories (Bar Harbor, Me.) were used at 6 to 10 weeks of age. Mice were housed five per cage and allowed free access to water and, initially, to D11 mouse chow. Following a one week acclimation period, mice were switched to a powdered diet (Mouse Breeder/Auto-JL K20 mouse chow, PMI Feeds, Inc., St. Louis, Mo.) for three days and allowed to adapt to the diet prior to the start of the PDE9 inhibitor dosing period.

The PDE9 inhibitor compound (Compound A) was administered in powdered mouse chow that was custom ground (Research Diets, Inc., New Brunswick, N.J.) as a compound/chow admixture; compounds were mixed with the chow to achieve consumption of the specified doses ranging from 1-200 mg/kg/day. In addition to a compound-free control group, a group consuming darglitazone (1 mg/kg/day) was also included as a positive control.

Mice were randomly assigned to groups of ten with five mice per cage. Body weight was determined on Day 0 and weekly thereafter. On Day 1, retro-orbital blood samples were obtained and plasma glucose was determined as previously described. On the final day of the study, blood samples were taken for glucose, triglyceride, insulin, and cGMP measurements, as previously described.

### Results

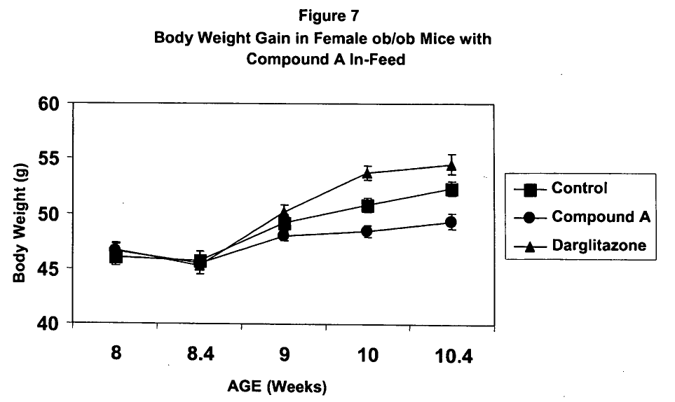

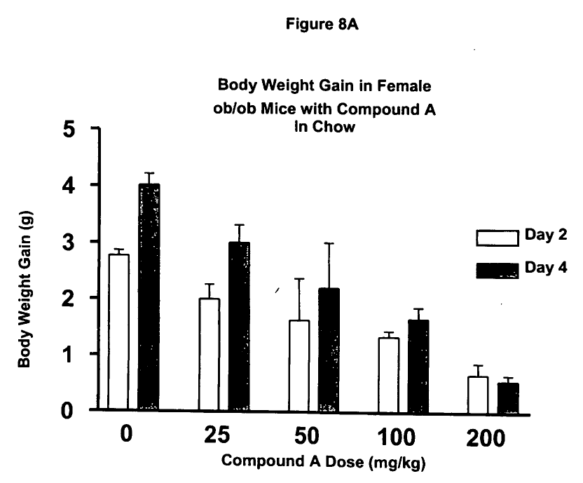

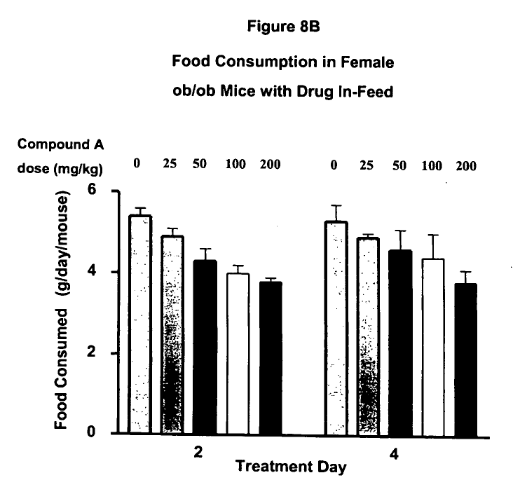

The results below represent results from several separate studies using the same above-described protocol. FIG. 7 shows a reduced body weight gain in ob/ob mice fed 100 mg/kg/day of the PDE9 inhibitor Compound A as compared to the mice fed either a compound-free control diet or a darglitazone-treated diet. Compound A elicited a dose-dependent effect following 2 and 4 days of treatment, both in terms of reducing the normal body weight gain (FIG. 8A) and also in terms of reducing food intake (FIG. 8B). The PDE9 effect on food intake could be transient, given that no effect on food intake was observed in the later stages of the study (FIG. 9) with the intermediate dose of 100 mg/kg/day.

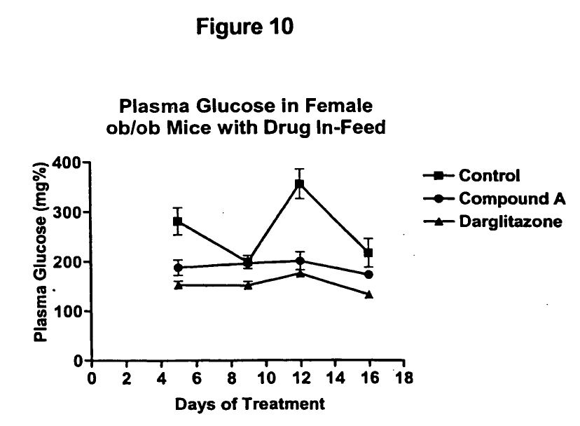

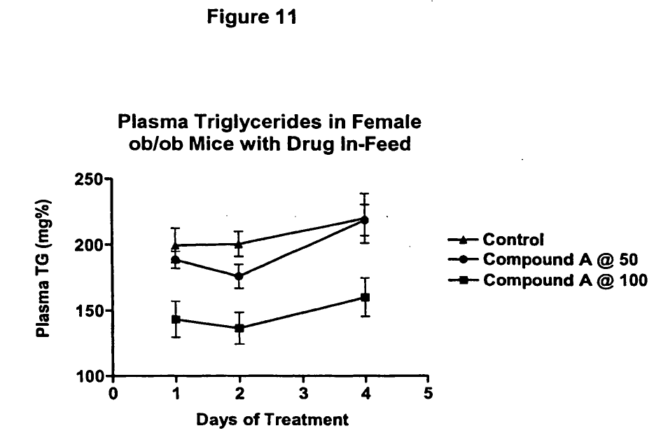

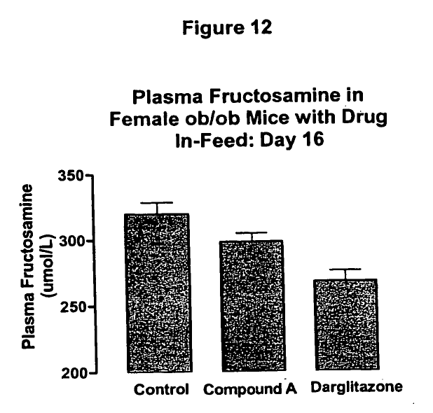

The intermediate dose of 100 mg/kg/day of Compound A also resulted in decreased glucose, triglycerides and fructosamine. Representative results are shown in FIG. 10, FIG. 11, and FIG. 12, respectively.

Both Examples demonstrate that causing a decrease in PDE9 activity is an effective method to reduce body weight and/or body fat, and can be used, e.g., to treat animal patients that are overweight, obese, or suffer from an eating disorder, and can be used in animal food stock species to produce leaner meat.

1. A method of treating an animal to reduce body fat comprising administering to an animal in need thereof a therapeutically effective amount of a phosphodiesterase 9 (PDE9) inhibitor.

2. A method of claim 1, wherein said mammal is overweight.

3. A method of claim 1, wherein said mammal is obese.

4. A method of claim 1, wherein said PDE9 inhibitor is a PDE9 selective inhibitor.

5. A method of treating an animal for an eating disorder, said method comprising administering to an animal in need thereof a therapeutically effective amount of a PDE9 inhibitor.

6. A method of claim 5, wherein said PDE9 inhibitor is a PDE9 selective inhibitor.

7. A genetically-modified mouse, wherein said mouse is homozygous for disruption of the PDE9 gene and wherein said mouse, following a six week high fat diet, exhibits reduced body weight or reduced fat mass in an adipose depot, as compared to a wild type mouse following a six week high fat diet.

8. A mouse of claim 7, wherein said mouse expresses an exogenous reporter gene under the control of the regulatory sequences of said PDE9 gene.

9. A mouse of claim 7, wherein said mouse exhibits nondetectable PDE9 activity.

10. A genetically-modified cultured mammalian cell, wherein said cell is homozygous for disruption of the PDE9 gene and wherein said cell, or a progeny cell derived from said cell, exhibits nondetectable PDE9 polypeptide activity wherein said cell or progeny cell would exhibit PDE9 polypeptide activity absent said homozygous disruption.

11. A genetically-modified mammalian cell of claim 10, wherein said cell is an embryonic stem (ES) cell.

12. A genetically-modified cell of claim 11, wherein said cell is a murine ES cell.

13. A genetically-modified cell of claim 11, wherein said cell is a human ES cell.

14. A method for producing the mouse of claim 7 comprising:
(a) introducing a DNA sequence into a mouse ES cell, wherein the DNA sequence comprises a PDE9 gene targeting construct, which, upon recombination with the PDE9 gene, disrupts the PDE9 gene;
(b) selecting a mouse ES cell whose genome comprises a disruption of the PDE9 gene;
(c) introducing an ES cell selected in step (b) into a mouse blastocyst or morulae;
(d) transplanting the blastocyst or morulae of step (c) into a foster mother mouse;
(e) developing the transferred blastocyst or morulae to term to produce a chimeric mouse; and
(f) obtaining a mouse homozygous for the PDE11 gene disruption by breeding chimeric mice of step (e) and mice heterozygous for the PDE9 disruption;
wherein said mouse homozygous for disruption of the PDE9 gene, following a six week high fat diet, exhibits reduced body weight or reduced fat mass in an adipose depot, as compared to a wild type mouse following a six week high fat diet.

15. An isolated nucleic acid molecule comprising a PDE9 gene targeting construct, wherein, upon recombination with the PDE9 gene, said construct disrupts the PDE9 gene.

16. A method of treating an animal to reduce food intake comprising administering to an animal in need thereof a therapeutically effective amount of a phosphodiesterase 9 (PDE9) inhibitor.

17. A method of claim 16, wherein said mammal is overweight.

18. A method of claim 16, wherein said mammal is obese.

19. A method of claim 16, wherein said PDE9 inhibitor is a PDE9 selective inhibitor.


SEQUENCE LISTING (3 sequences)

SEQ ID NO: 1 - DNA, 200 bp, Mus musculus
ttgagggagaacctctcactggcctgggtgaccagcgagtacatgagttctagggactgaacttgggtcatcactcttaggtaggaagcacattactccctgagccaactctccagctcaacttctgtgttttgcaggtggttttcagcaagtactgcaactccagtgacatcatggacctgttctgtatcgccaccggc

SEQ ID NO: 2 - DNA, 200 bp, Mus musculus
cagacgacgccatggtctccatcgatcccaccatgcctgcgaattcagagcggtaagcgactcctcctggggtgtctcacttgtctatgatgtgtgccagggagtctgcaatttggctttgtttactactgctaagacgtgtgaagtttcagcagaagagggaaggaggcagatgggaggcaggcggaggcttggttcac

SEQ ID NO: 3 - DNA, 1929 bp, Mus musculus
gcgggcgcgatgggggccggctcctcaagctaccggcccaaggccatctacctagacatcgatggacgcatccagaaggtggttttcagcaagtactgcaactccagtgacatcatggacctgttctgtatcgccaccggcctgcctcggaacaccaccatctcccttttaaccacagacgacgccatggtctccatcgatcccaccatgcctgcgaattcagagcgcactccctacaaggtgagacctgtggctgtgaagcaagtgtctgaacgagaagaacttatccagggcgtgctggcccaggtggcggaacaattttccagagcgtttaagatcaacgagctgaaagccgaagttgcaaatcacctggccgtgctggagaaacgggtggaattggaaggacttaaagtggtggagatcgaaaaatgcaagagtgacattaaaaagatgcgggaggagttggcagctaggaacagcaggaccaactgtccatgtaaatacagttttttggataacaagaagttgacacctcgacgtgatgtccccacttaccccaagtacctgctctccccagagaccatcgaagccctacggaagcccacctttgatgtctggctttgggaacccaacgagatgctgagctgtttagaacatatgtaccacgaccttggtctggtcagggacttcagcatcaacccaatcacgctccgcaggtggctgctctgtgtgcatgacaactacaggaacaaccccttccacaacttccggcactgcttctgtgtgacacagatgatgtacagtatggtctggctctgtggcctccaggagaagttttcccagatggacatcttggtcctgatgactgcagccatctgccatgacctggaccacccagggtacaacaatacataccagatcaatgcccgcacggaactcgctgtgcgctacaacgacatctcaccgctggagaaccaccactgcgccattgccttccagatcctggcaagacctgagtgcaacatcttcgccagtgtgccacccgagggcttcaggcagatcaggcaggggatgatcacattgatcctggctaccgacatggcaaggcatgctgaaattatggattctttcaaagaaaagatggagaattttgactacagcaacgaggagcacctgaccctgctgaagatgattctcataaaatgctgtgatatctccaatgaagtccgtcccatggaggtggcagaaccgtgggtggactgtttactggaagaatattttatgcagagtgaccgtgagaagtccgaaggccttcctgtggccccattcatggaccgagacaaagtgaccaaagcaacagcccaaattgggttcatcaagtttgtcctgatcccaatgtttgaaacagtgaccaagctcttccccgttgttgaggagaccatgctgcggccgctctgggagtcccgagaacactacgaggagctgaagcagctggacgatgccatgaaggagttgcagaagaagacagagagcttaacatctggggccccggaaaacaccacagagaagaacagagatgcaaaagacagtgaaggccattctcctccaaactgatggacatctcaatgtgtgcacagactgtaccagttagagcagatgaattgtggcctgtgagtggacagagccaagcgaggcttcccaggatcttccacacaaggatggtcacgcccagacaaccctgatgacctgctacagaccatgttttctaagaaccattttgttccctgatataaaaacaggaatcccgatgctgttcagaatttttatttttaaactgttttttaaataatatatttttacacagaaaaaaaaaaaaaaaaaaaaaaaaaaaaaaaaaaaaaaaaaaaaaaaaaaaaaaaaaaaaaaaaa